(19) United States
(12) Patent Application Publication
(10) Pub. No.: US 2026/0198933 A1
(43) Pub. Date: Jul. 16, 2026

(54) OPTICAL TRANSMISSION MICROCATHETER DEVICES AND METHODS THEREFOR

(71) Applicant: NORTHERN VASCULAR SYSTEMS INC., Etobicoke (CA)

(72) Inventors: Mark HARDUAR, Brampton (CA); Yuta DOBASHI, Vancouver (CA); Joel RAMJIST, Toronto (CA); Konrad WALUS, Delta (CA); Victor YOUNG, North York (CA)

(21) Appl. No.: 19/135,527

(22) PCT Filed: Dec. 6, 2023
(86) PCT No.: PCT/CA2023/051616

§ 371 Date: Jun. 4, 2025

Related U.S. Application Data
(60) Provisional application No. 63/386,279, filed on Dec. 6, 2022.

(51) Int. Cl. A61B 17/12; A61B 17/00; A61M 25/00

(52) U.S. Cl. CPC A61B 17/12195; A61M 25/0021; A61B 2017/00061; A61B 2017/00938; A61M 2025/0042

(57) ABSTRACT

Optical transmission microcatheter devices and methods of endovascular embolization are described herein. There is provided a microcatheter device comprising a working lumen and a mechanically protected waveguide. The working lumen has at least one outlet port at a distal end of the working lumen and is configured to provide intravascular injection of a Photoactivated Embolic Agent (PEA) to a target vessel via the at least one outlet port. The mechanically protected waveguide (MPW) is operably connected to the working lumen and is configured to emit at least one electromagnetic wave via the at least one outlet port for delivery to the target vessel.

### CROSS-REFERENCE TO RELATED APPLICATIONS

This application claims priority to U.S. provisional Patent Application No. 63/386,279 filed on Dec. 6, 2022, the contents being incorporated herein by reference.

### FIELD

The present disclosure generally relates to the design of endovascular microcatheters. In some aspects, the present disclosure relates to the methods for therapeutic embolization by way of injecting a photoactivated embolic or therapeutic agent.

### BACKGROUND

Endovascular embolization is a minimally invasive catheterization procedure that aims to cease the local blood flow in peripheral vasculature to address pathologies including hypervascular tumors, arteriovenous malformations, subdural hematomas, and aneurysms. From a puncture of a superficial vessel, diseased blood vessels are often difficult to reach via open surgeries can be accessed by a catheter, where a chosen flow control device or agent is deployed. Endovascular embolization when used as a primary or adjunct method of disease management can significantly reduce perioperative risks and patient recovery time.

Current embolic devices and agents clinically available include mechanical devices such as detachable coils, flow diverters, precipitating liquid polymer agents, and suspended particulate materials. Metallic coils for aneurysm treatments are prone to recanalization due to insufficient filling of the aneurysmal cavity arising from the device compaction. Flow diverters, which have more recently become a popular choice for aneurysm treatments, are prone to malapposition against the parent arterial wall leading to residual aneurysmal perfusion. Flow diverters also put patients on lifelong dual antiplatelet therapy. Uncontrolled solidification seen in existing injectable embolic agents such as Onyx (Medtronic™) has resulted in off-target embolization leading to ischemic episodes and entrapped catheters which can dissect healthy vessels and cause rupture.

Commercially available neurovascular microcatheters used for endovascular embolization such as the SL10 (Stryker), and the Sceptor XC (Balt), are extremely low profile (typically less than 1 mm outer diameter) and have high compliance, only requiring ~1 gram-force for bending of the distal end. This allows the deployment of embolics in highly complex locations within the neurovascular network. Trade-offs between dimensions, navigability, softness, and infusion pressures are often nontrivial in microcatheter design. Microcatheters are often precluded from gaining additional features which may be available in larger dimensions, such as pull wires for active steering. The limited capabilities of microcatheters beyond infusion and balloon occlusion have been prohibitive of innovation in novel embolics. For instance, balloon-assisted deployment of the Onyx HD-500 precipitating liquid embolic agent in cerebral aneurysms saw limited adoption possibly due to the lack of deployment control, with several case reports of off-target embolism in the literature.

There is a clinical need for devices and methods to enable endovascular embolization with i) a precise spatiotemporal embolic deployment control ii) a high degree of shape conformity to a variety of vascular morphologies and iii) superior mechanical properties of the embolic, free of structural compaction or breakage, to minimize the reperfusion of the treated vessels or other iatrogenic events.

### SUMMARY

According to some implementations, there is provided a microcatheter device comprising a working lumen and a mechanically protected waveguide. The working lumen has at least one outlet port at a distal end of the working lumen and is configured to provide intravascular injection of a Photoactivated Embolic Agent (PEA) to a target vessel via the at least one outlet port. The mechanically protected waveguide (MPW) is operably connected to the working lumen and is configured to emit at least one electromagnetic wave via the at least one outlet port for delivery to the target vessel.

According to some implementations, the mechanically protected waveguide is: at least partially embedded in a wall of the working lumen; co-extruded into the wall of the working lumen; at least partially separated from the working lumen by at least one barrier; coupled to an exterior section of the working lumen; woven into a braided portion of the microcatheter; and/or at least partially disposed within an enclosure separate from the working lumen.

According to some implementations, the microcatheter device further comprises a pre-cure region within the microcatheter and proximate to the at least one outlet port, the pre-cure region being configured to regulate the viscosity of the PEA prior to injection into the target vessel.

According to some implementations, the mechanically protected waveguide comprises a multimode optical fiber.

According to some implementations, the working lumen comprises a biocompatible material, such as Nylon, Vestamid, Pebax and PTFE.

According to some implementations, the mechanically protected waveguide is configured to emit the electromagnetic wave to the target vessel and/or the pre-cure region.

According to some implementations, the microcatheter device further comprises at least one beam forming element. According to some implementations, the at least one beam-forming element comprises one or more optical components, such as one or more of a lens, a diffuser, a filter, a reflector and a mask. According to some implementations, the at least one beam-forming element is configured to provide at least one radial emissive beam for delivery to the pre-cure region.

According to some implementations, the microcatheter device further comprises at least one emissive beam which comprises: a first beam directed towards the target vessel, and a second beam directed towards the pre-cure region.

According to some implementations, the at least one beam-forming element comprises a plurality of beam-forming elements configured to provide a plurality of radial beams in a plurality of directions.

According to some implementations, the mechanically protected waveguide comprises a Fiber Bragg Grating (FBG). According to some implementations, the FBG element is tuned to a secondary wavelength and configured to back-reflect light having the second wavelength. According to some implementations, the FBG is configured to selectively propagate light of a first wavelength in a first direction and to propagate light of a second wavelength in a second direction based on a frequency of the electromagnetic wave. According to some implementations, the first direction is towards the at least one outlet port and the second direction is a radial direction.

According to some implementations, the microcatheter device further comprises a detachment mechanism configured to detach solidified PEA from a distal end of the microcatheter. According to some implementations, the detachment of the PEA is induced by emitting through the MPW a predetermined wavelength of electromagnetic waves configured to photodegrade the crosslinked PEA. According to some implementations, the detachment mechanism comprises an ultra-hydrophobic coating applied to an exterior surface of the distal end proximate the at least one outlet port.

According to some implementations, at least a portion of the distal end comprises a reflective coating and/or reflective material and is configured to reflect excess light emitted radially outward in a radially inward direction.

According to some implementations, the microcatheter device further comprises an optical cavity operatively connected to the pre-cure region.

According to some implementations, the mechanically protected waveguide comprises a fiber optic configured to direct at least one evanescent wave to be in physical contact with the PEA.

According to some implementations, the optical index of refraction can be detected in the PEA. According to some implementation, the microcatheter further comprises a detecting mechanism configured to detect changes in the optical index of refraction of a medium surrounding the mechanically protected waveguide.

According to some implementations, the microcatheter device further comprises a power injector configured to provide a fluid to the outlet port at a pre-determined pressure and to determine changes in a backpressure of the fluid. According to some implementations, the determination of changes in back pressure of the fluid is made in real-time.

According to some implementations, the microcatheter device further comprises a detachable tip. According to some implementations, the detachable tip is configured to dissolve within the target vessel over a predetermined amount of time (typically within tens to hundreds of seconds). According to some implementations, the detachable tip is biocompatible.

According to some implementations, a distal end of the mechanically protected waveguide is free to float within the pre-cure region.

According to some implementations, the microcatheter device further comprises at least one valve mechanism.

According to some implementations, the distal end of the working lumen is tapered. According to some implementations, the taper is sized to provide a desired flow velocity of the PEA to the target vessel.

According to some implementations, the microcatheter device further comprises a solid element inserted into the working lumen.

According to some implementations, the microcatheter device further comprises at least one additive liner within the pre-cure region, the at least one additive liner being configured to limit adhesion of cured PEA to the inner and/or outer surface of the working lumen.

According to some implementations, there is provided a method of endovascular embolization comprising: introducing the microcatheter device according to any one of the preceding implementations to the target vessel; injecting the Photoactivated Embolic Agent (PEA) into the target vessel until a desired volume of the target vessel has been filled; and after or simultaneously with the injecting, emitting electromagnetic wave to the PEA disposed in the target vessel at a solidification wavelength to the PEA. According to some implementations, the emitting comprises the electromagnetic wave to the pre-cure region.

According to some implementations, the method further comprises, prior to emitting the electromagnetic wave at the solidification wavelength, determining whether there has been a failure of the microcatheter device. According to some implementations, determining whether there has been a failure of the microcatheter device comprises sensing at least one electromagnetic wave back-reflected from the PEA.

According to some implementations, the method further comprises, after emitting the electromagnetic wave at the solidification wavelength, removing the microcatheter from the solidified PEA and the target vessel. According to some implementations, removing the microcatheter comprises detaching the microcatheter from the solidified PEA. According to some implementations, detaching the microcatheter comprises activating a photodegradation mechanism.

According to some implementations, the method further comprises determining a solidification state of the PEA. According to some implementations, the determining is preformed in real-time or near real-time. According to some implementations, the determining comprises detecting at least one electromagnetic wave back reflected from the PEA disposed in the target vessel.

### BRIEF DESCRIPTIONS OF THE DRAWINGS

For a better understanding of the various implementations or embodiments described herein and to show more clearly how they may be carried into effect, reference will now be made, by way of example only, to the accompanying drawings in which:

FIGS. 1a to 1f depict some example vascular morphologies amenable to embolization using the optical transmission microcatheter device (100) and photoactivated embolic agent (109).

FIG. 2a depicts example microcatheter (100) with a working lumen (101) for delivery of a Photoactivated Embolic Agent (PEA) and within an example target vessel (200, 203, 204, or 205), according to non-limiting implementations. A mechanically protected waveguide (MPW) (102) is embedded in the wall of the working lumen (101). As well, the distal end (134) of the microcatheter has an outlet port (113) where the PEA (109) is injected into the target vessel, according to non-limiting implementations.

FIG. 2b depicts an example embodiment of microcatheter where the MPW (102) is configured such that a region within the working lumen (101) is exposed to electromagnetic waves (104), according to non-limiting implementations.

FIG. 2c depicts an example embodiment where the MPW (102) is actively transmitting an electromagnetic wave (104), towards the PEA (109), according to non-limiting implementations.

FIGS. 3a-3h depict several iterations of how the mechanical protection is applied to the MPW (102), according to non-limiting implementations.

FIG. 4 depicts multiple beam-forming elements (105) directing electromagnetic waves (104) towards the pre-cure region (103) and through the outlet port (113), according to non-limiting implementations.

FIG. 5 depicts multiple beam-forming elements (105) directing electromagnetic waves radially inwards and/or outwards (106) of the working lumen, according to non-limiting implementations.

FIG. 6a depicts multiple beam-forming elements (105) directing electromagnetic waves (106 and/or 104) radially inwards, outwards, and/or in the same direction of the outlet port (113), according to non-limiting implementations. FIG. 6b depicts an example of how multiple beam-forming elements (105) can be arranged with a MPW (102), according to non-limiting implementations.

FIG. 7 depicts a Fiber Bragg Grating (FBG) element (108) positioned between emitting electromagnetic waves (104, 114), according to non-limiting implementations.

FIG. 8a shows the microcatheter (100) where the cured PEA (111) has been fully and/or over-cured to the microcatheter (100) within the target vessel and is not easily removed, according to non-limiting implementations. FIG. 8b-1 depicts an emitting beam intended to soften/dissolve cured PEA (116), according to non-limiting implementations. FIG. 8b-2 depicts the softened region of the cured PEA (117) which allows for the separation of the microcatheter (100) and the bonded PEA (115), according to non-limiting implementations.

FIG. 9 depicts an example microcatheter with the distal end being coated with a coating or liner (118), according to non-limiting implementations, which limits the bond between the microcatheter (100) and the cured PEA (111). This coating can be hydrophobic or hydrophilic.

FIG. 10 depicts an example microcatheter where the distal end is comprised of reflective material (119), according to non-limiting implementations.

FIG. 11a depicts an example microcatheter emitting an electromagnetic wave (104) with the intent of viscosifying uncured PEA (109), according to non-limiting implementations. FIG. 11b depicts an example microcatheter in which the PEA (111) us fully cured and the reflected electromagnetic waves from cured PEA (112) channeling into the MPW (102).

FIG. 12 depicts an example microcatheter where the MPW (102) is in direct contact with the PEA (109), according to non-limiting implementations. An evanescent wave source (120) is propagating. An evanescent wave (120) and a reflected evanescent wave source are created (122).

FIG. 13 depicts a power injector capable of inferring backpressure (123) hydraulically connected to the working lumen (101), according to non-limiting implementations.

FIG. 14 depicts a power injector capable of inferring backpressure (123) hydraulically connected to the working lumen (101), according to non-limiting implementations. A fault or leakage (124) occurs within the hydraulic system (in this case the working lumen (101).

FIG. 15 depicts an example microcatheter where the MPW (102) has a failure and/or breakage (130) along its length, according to non-limiting implementations, the MPW (102).

FIG. 16 depicts an example microcatheter with a detachable tip (132) mounted at its distal end, according to non-limiting implementations.

FIG. 17 depicts a detailed example flow chart (400) of how the PEA is used with the microcatheter, according to non-limiting implementations.

FIG. 18a depicts an example microcatheter where an outlet relief valve (135) is attached to the outlet port of the working lumen, according to non-limiting implementations. FIG. 18b depicts a scenario where the PEA is cured to a solid, according to non-limiting implementations. Pressure within the working lumen forces the cured PEA through the outlet relief valve (also referred to herein as an outlet port valve).

FIG. 19 depicts the working lumen material acting as a diffuser for the electromagnetic wave, according to non-limiting implementations.

FIGS. 20a and 20b depict pre-cure regions that have been widened (137). to assist with changing the flow velocity and increasing the area for exposure to electromagnetic waves, according to non-limiting implementations.

FIGS. 21a and 21b depict non-limiting configurations of the pre-cure regions with tapered or narrowing wall, resulting in a smaller cross-sectional area at the tip.

FIGS. 22a and 22b depict simplified example flow charts (300a and 300b) outlining the use of the microcatheter device to deposit a PEA, according to non-limiting implementations.

FIG. 23 depicts an example of multiple MPWs (102) housed within respective channels and terminated at various positions for multiple beam directions, according to non-limiting implementations.

### DETAILED DESCRIPTION

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the implementations described herein. However, it will be understood by those of ordinary skill in the art that the implementations described herein may be practiced without these specific details. In other instances, well-known methods, procedures, and components have not been described in detail so as not to obscure the implementations described herein. Furthermore, this description is not to be considered as limiting the scope of the implementations described herein in any way, but rather as merely describing the implementation of the various implementations described herein.

Ideally, the existing limitations in the medical devices and materials clinically available for endovascular embolization are addressed by a multifaceted solution which addresses the challenges related to the embolics as well as their applicators (i.e. the microcatheters). One solution may be for the embolic material to be stimuli responsive such that the solidification process can be triggered on-demand, while the catheter may possess the ability to trigger such solidification process.

Described herein are optical transmission microcatheter devices and endovascular injection method of photoactivated embolic agents.

The present application generally provides a means of endovascular embolization to cause a controlled cessation of the local blood flow in the context of various vascular diseases. One aspect of the systems and methods described herein relates to a microcatheter design which can deliver a Photoactivated Embolic Agent (PEA) while simultaneously emitting electromagnetic waves through a Mechanically Protected Waveguide (MPW) to target vascular anatomy, such as a target blood vessel, and/or internal to the microcatheter device.

In one aspect, the properties of the PEA are such that the material is initially in a flowable liquid state and once it is exposed to an electromagnetic radiation of predetermined wavelengths (typically 200~600 nm), it solidifies to form a soft elastic solid. Thus, one purpose of the ability of the optical transmission microcatheter device to emit an electromagnetic radiation is to controllably trigger the solidification of the PEA to form an embolic plug at a desired location within diseased vasculature.

According to some implementations, the PEA is a yield stress fluid. In its rest state, the PEA may exhibit a solid-like property while it can enter a flowable state upon being subjected to a certain stress such as an infusion pressure across a catheter lumen. In some instances, such properties of the PEA may enable ease of PEA injection through a microcatheter while simultaneously providing initial resistance to blood flow once it enters the vasculature by recovering its solid-like state. This initial resistance to blood flow may improve the efficacy of photoactivated solidification by preventing wash-out of the material. Subsequent photoactivated solidification of the PEA is typically irreversible and further stabilizes the injected material in an endovascular cavity.

The microcatheter is comprised of a working lumen capable of delivering the PEA to the target vessel using, according to some implementations, a syringe, which is either hand-driven or driven by an infusion pump. The microcatheter is typically navigated to the target vessel with the use of a guidewire. PEA is then injected through the microcatheter and into the target vessel through the outlet port of the microcatheter. Deposited PEA into the target vessel can be exposed to electromagnetic waves to initiate the crosslinking, increasing the viscosity and modulus of the PEA and eventually transitioning to a soft elastic solid material. One attribute of the microcatheter is that the waveguide is mechanically protected (MPW), otherwise use of accessories such as guidewires and high-pressure hydraulic injection will damage the waveguide and deposit electromagnetic waves in an unintended location.

According to some implementations, within the distal portion of the microcatheter comprises a pre-cure region. Prior to the PEA exiting the microcatheter and entering the target vessel, the pre-cure region can have exposure to electromagnetic waves which allows for viscosification or solidification of the PEA. There is a wide variety of target vessels that the operator may be occluding, including but not limited to high flow regions or regions wherein over-penetration of the vasculature must be avoided due to the critical structures present downstream. Pre-curing the PEA can better localize the embolic deposition either by remaining tethered to the catheter tip or by way of size exclusion when the solidified mass becomes entrapped in distal vasculature, that is typically smaller in size. For example, if the target vessels are relatively large with high blood flow, a higher viscosity PEA would usually be suitable, while if the target vessels are relatively narrow and low flow rate, a lower viscosity or shape-conformal PEA would usually be recommended. The ability to control the gives a level of control on the PEA to the operator.

There are several methods for providing mechanical protection to the waveguide contemplated. These methods include, but are not limited to, i) partially embedded the MPW into the working lumen, ii) co-extruding the working lumen wall with the MPW, iii) having a barrier between working lumen and MPW, iv) coupling the MPW to the exterior of the working lumen, v) weaving the waveguide between braiding layers, vi) protected separately from the working lumen, and/or vii) partially embedding the MPW into the wall of the working lumen while allowing a region to physically move into the working lumen However, it is understood that any suitable means or combination of means for mechanically protecting the waveguide is contemplated.

The MPW can be configured in several different ways to emit electromagnetic waves. For example, the electromagnetic waves can be emitted towards the outlet port of the microcatheter, through the outlet port towards the target vessel, towards the pre-cure region, and/or radially towards the target vessel. This can be achieved by the MPW placement within the working lumen. Additionally, beam-forming elements (such as micro-optical components) could be added anywhere suitable along the optical path of the MPW (or distal to the MPW) to further assist with directing the electromagnetic waves toward any of the target regions. The use of beam-forming elements are typically necessary for complex beam profiles and directions.

According to some embodiments, a Fiber Bragg Grating (FBG) is added along the length of the MPW. This addition generally provides a means to select a particular electromagnetic wave for a particular direction. The FBG is tuned such that it will reflect a specific bandwidth of electromagnetic waves and the remaining spectrum of electromagnetic waves will be transmitted through. An example use of the FBG is to orient the FBG between 2 different beam-forming elements. If a bandwidth matching the reflection profile of the FBG is selected, all beam-forming elements proximal to the FBG will emit electromagnetic waves. If a bandwidth not matching the reflection profile of the FBG it will be transmitted through. This mechanism generally gives a further sense of control to the operator. A foreseeable application is if the operator wished to independently change the incident power of the electromagnetic waves in the pre-cure region with respect that of the outlet port.

According to some embodiments, making use of multiple wavelengths, a primary wavelength of electromagnetic wave will crosslink and viscosify or solidify PEA. A secondary wavelength can be used to cleave the crosslinks and liquify the solidified certain types of PEA. This is helpful if the microcatheter has become stuck with the PEA and cannot be retrieved while engaged in the vessel. Alternatively, an hydrophobic coating can be applied to the distal end of the microcatheter to ensure strong bonding of cured PEA and the microcatheter does not occur.

According to some embodiments, multiple MPWs are integrated in order to achieve various electromagnetic energy distributions. For instance, in a two-MPW configuration, it is possible to have one MPW terminate at the proximal side of the pre cure region while the other MPW is continuous to the tip of the catheter. Such a configuration The MPWs can be operated either sequentially or in concert.

In an ideal operation of the pre-cure segment of the catheter, a predetermined level of electromagnetic energy, typically a range of about 1 to about 1000 mJ at a suitable wavelength (typically about 200~about 600 nm), should reliably viscosify or solidify the flowable PEA into a soft solid, regardless of the variation in the infusion forces the user may exert during use. In other words, according to some implementations, the system needs to be sufficiently insensitive to such input variations so as not to inadvertently release uncured materials (in the case of excessively rapid infusion) or clog the pre-cure region (in the case of excessively slow infusion), either of which can lead to procedural complications.

Furthermore, the ability of the pre-cure region in conjunction with the electromagnetic energy source to continuously viscosify or solidify the injected PEA could be independent of catheter placement within a given vasculature or local hemodymanics. For instance, the catheter tip may not necessarily lay in parallel to a blood vessel but rather be pointed against the wall. Such a confined positioning of the catheter may disturb the flow of the PEA out of the catheter port and affect the solidification process.

According to some embodiments, one or more valve mechanisms may be employed within the catheter. The valve may be pressure activated, limiting the passage of uncured PEA until a predetermined pressure differential (typically in the order or one tenth to hundreds of PSI) across a given segment of the catheter is attained. In one aspect, such a valve mechanism may be employed at the distal port of the catheter to prevent accidental release of uncured PEA (flowable under low pressures) while allowing cured PEA (flowable under high pressures) to be released and deposited into the target vasculature.

To prevent unintended release of uncured precursors, some embodiments may employ a tapered lumen wall on the distal end of the catheter. Such a tapered wall may generally reduce the flow velocity of the precursor over the pre cure regions, providing a greater exposure to the electromagnetic energy and ensuring a more robust viscosification or solidification. The tapered wall may also act to reflect forward-firing beams emitted by the waveguide in some embodiments towards the direction of the pre cure region, further enhancing the efficiency of the viscosification or solidification.

To increase the crosslinking efficiency within the pre-cure region, a reflective material can be added around the pre-cure region such that any radially emitting electromagnetic waves are reflected towards the pre-cure region. This can also direct a large portion of the incident electromagnetic waves towards the outlet port (and target vessel). The reflective material may be made from a radio-opaque material.

According to some implementations, there will be back-reflected electromagnetic waves from uncured PEA. As the PEA transitions from uncured to cured, the optical properties of the back-reflected electromagnetic waves can vary. The total power, spectral or polarization characteristics can change based on the PEA state. Assessing these EM Wave characteristics on the back-end can help inform the operator of the state of the PEA.

Similarly to monitoring back-reflected electromagnetic waves from the PEA, according to some implementations the evanescent wave power can be inferred by monitoring the reflected signal off of an FBG. This will also be an indirect way of informing the operator the state of the PEA. Further details are given in the detailed description.

If the PEA is contained in the hydraulic system of the microcatheter, when the PEA changes from a liquid to solid state, this can cause an increase in the backpressure of the system. Using a power injector capable of detecting pressure, the viscosity state of the PEA can be inferred. Conversely, if there is a break or fault in the hydraulic system, the backpressure would dramatically drop, and this will also be sensed by the power injector, according to some embodiments.

Another failure scenario is if the MPW has a break or imperfections inflicted on it. If any imperfections are induced within the optical path, this will create an increase in the return electromagnetic waves (known as return loss). Monitoring this for a sudden change is a method for inferring whether any damage has happened to the MPW.

According to some embodiments, the distal portion can have a detachable tip. Methods of detachment can include one or more of the following in any suitable combination: physically separating by force such that a pre-determined tear point is broken, physically separating by dissolving the pre-determined tear point by the emitted electromagnetic wave (or secondary electromagnetic wave), physically separating by dissolving the pre-determined tear point through the use of a biocompatible injectable gel specifically designed for dissolving. The detachable tip may be made of a biocompatible material that would dissolve as it would remain as an implantable device. A non-limiting example of such a material is polyvinyl alcohol (PVA) which is known to slowly dissolve in water and has previously been made into micronized particles and used as temporary embolics.

A typical use of the described microcatheter, according to some implementations, would include navigating the microcatheter to the treatment site through the use of a conventional guidewire. Pre-embolization scans of the diseases site would be performed using digital subtraction angiography. At this point the operator can decide what type of viscosity is necessary for the PEA to optimally fill the treatment site based on the disease type. The operator(s) would usually select the correct EM wave power for the desired viscosity and inject the PEA accordingly. The operator will usually inject the PEA to the disease until its filled. Using fluoroscopy, the operator can confirm when the treatment site is filled. The operator will perform a post injection cure of the PEA by emitting EM waves to the treatment site until the embolic plug is solidified. There are several methods describe herein to monitor the solidification of the PEA. After completing the solidification, the operator can remove the microcatheter from the treatment site. According to some embodiments, if there are difficulties in removing it due to adhesion between the catheter and the solidified PEA, the operator may activate a detachment mechanism discussed herein. According to some implementations, throughout the entire use of the microcatheter, there are some fault detection methods which are monitored. Faults include, but are not limited to, leakage from the catheter or a breakage in the MPW.

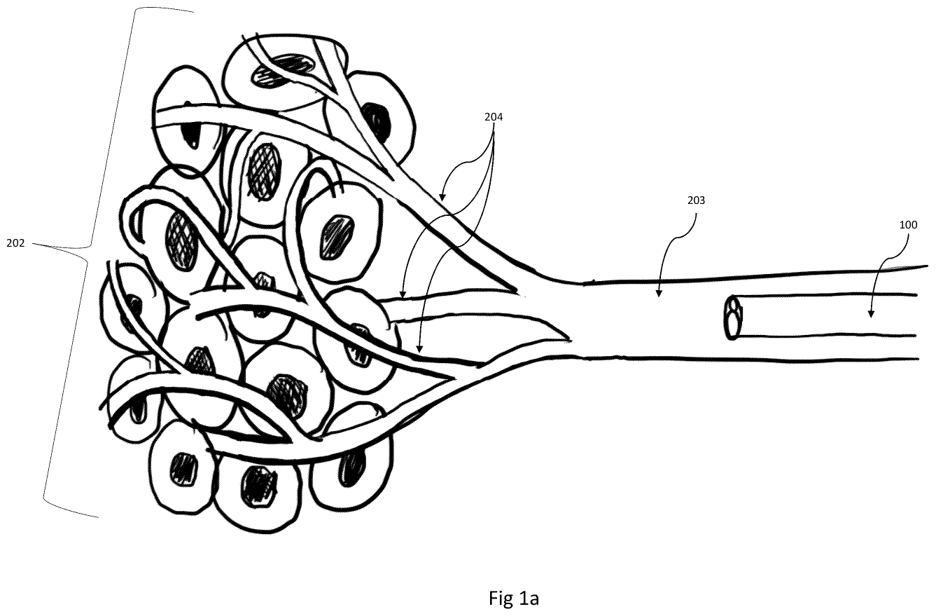

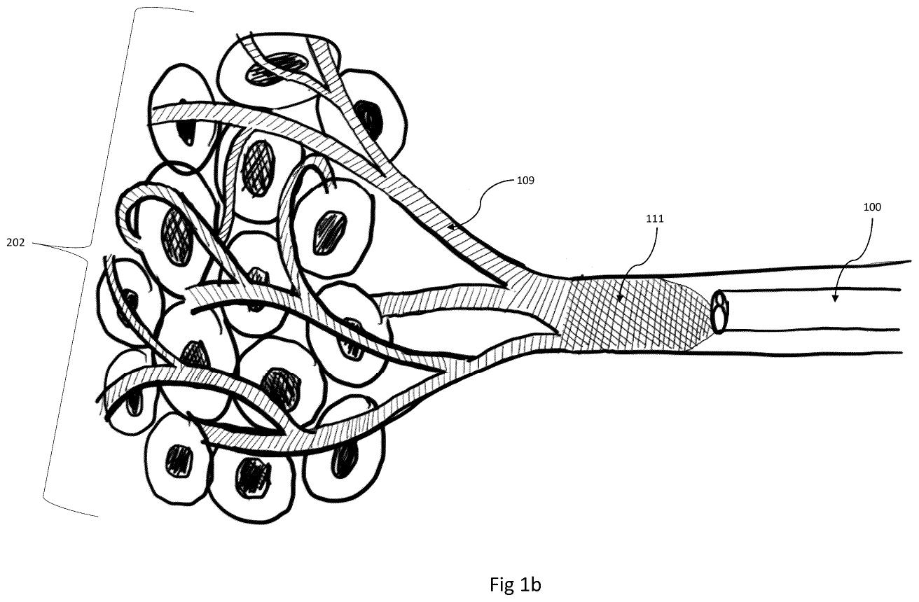

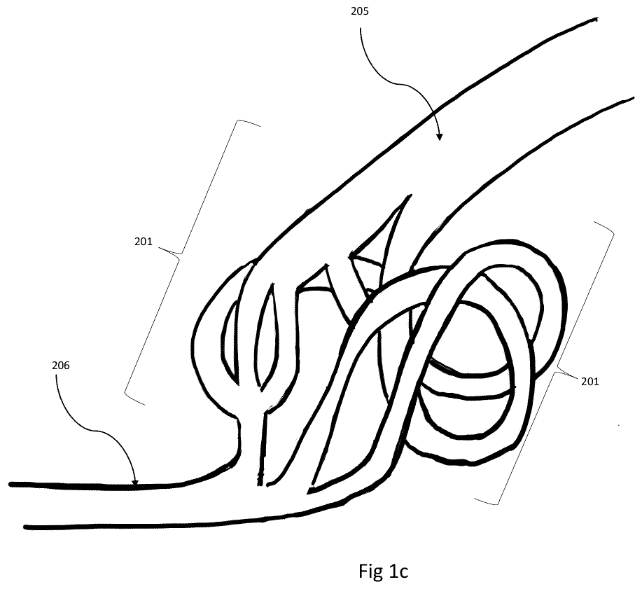

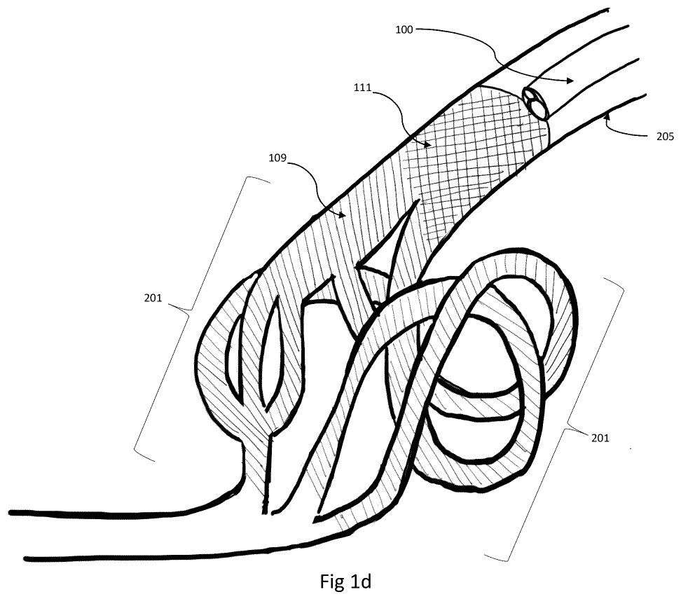

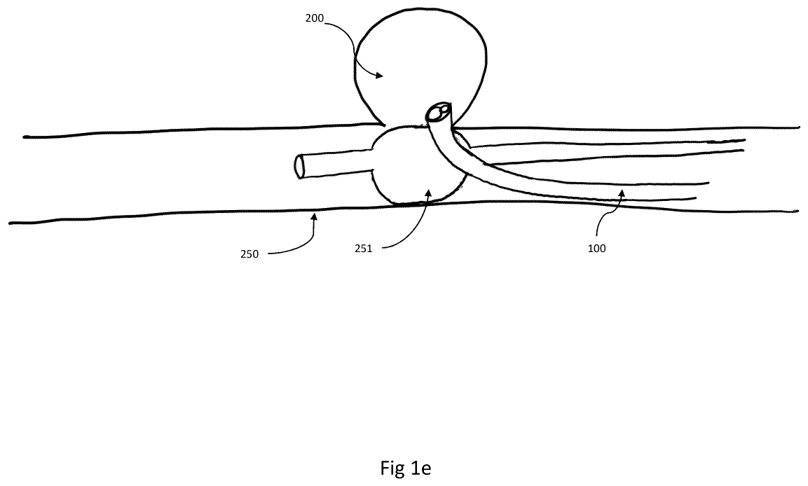

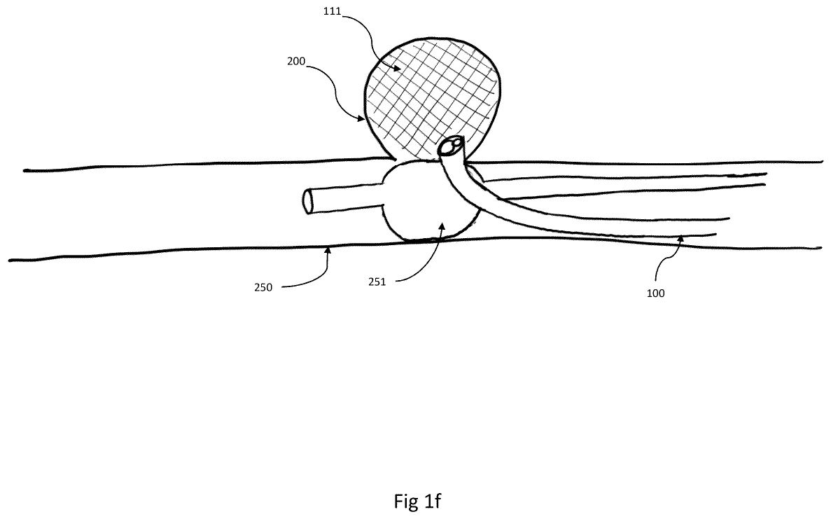

Attention is directed to FIGS. 1a to 1f, which depict the use of optical transmission microcatheter device (100) in combination with a photoactivated embolic agent (PEA) (109, 111) within example vascular morphologies (200, 201, 202), according to non-limiting implementations. In a given blood vessel, for example, the operator usually assesses the local vascular morphologies, such as the nominal blood flow rates, vessel diameter, and downstream anatomy, to determine the optimal injection scheme. Some non-limiting examples are provided in FIGS. 1a to 1f. The first example shown in FIGS. 1a and 1b is of a hypervascular tumor (202), which is a terminal vasculature comprised of capillary vessels (204). In this case, the PEA (109) is injected into the structure from a larger feeder artery (203) where the tip of the optical transmission microcatheter device (100) is positioned. Note here that throughout FIGS. 1a to 1f, the line-shaded regions represent uncured PEA (109) while the cross-hatched regions represent the photoactivated and solidified PEA (111). Due to the inherent rheological properties of a PEA (109), which may include shear recovery, the material released into the microvasculature is reasonably expected to form a plug up to the point of injection. An example shown in FIG. 1b, the operator may choose the inject the precursor PEA (109) into the lesion initially without the electromagnetic wave emission (i.e., no viscosification or solidification) to achieve a distal penetration. The proximal portion of the injected PEA is then photoactivated (111), generally making the entire structure is stable. The next example as shown in FIGS. 1c and 1d is of an arteriovenous malformation (201), which is a high-pressure shunting lesion between arteries (205) and veins (206) comprising of many interweaving nidus vessels (201). In this case, because the structure is not terminal and drains into venous network (206), excessive penetration of the PEA (109) can cause downstream ischemic events. The operator may address this situation in several ways. One way is to perform a temporary balloon occlusion proximal to the injection point such that the PEA (109) injected is under a reduced hemostatic pressure driving it distally, improving operator control of infusion pressure and penetration depth. Another approach is to simply preselect a higher viscosity PEA (109), provided that it is injectable through the optical transmission microcatheter device (100), such that it is more resistant to excessive flow into distal vasculature. Alternatively, one may simultaneously inject the PEA (109) and emit predetermined electromagnetic radiation to the PEA (109) as it enters the blood vessels, thereby either viscosifying or solidifying the PEA (111). This approach may simultaneously benefit from the lower infusion pressure and higher occlusive capacity. Note that the effective distribution of uncured PEA (109) and photoactivated PEA (111) occupying the nidus (201) can vary greatly depending on the injection technique chosen and it shall be understood that the FIG. 1d represents an exemplary case. For instance, should an operator choose to inject the uncured PEA (109) while emitting electromagnetic energy at all times, the entire structure could comprise of the photoactivated PEA (111). In the final example of an aneurysm shown in FIGS. 1e and 1f, the embolic material must be strictly confined within a small aneurysmal space (200) without leakage into the parent artery (250), the operator may wish to embolic employ a balloon catheter (251). The injection of PEA and electromagnetic wave emission into the aneurysmal sac (200) may be performed either in sequence or concurrently to seal the structure, as can be appreciated that photoactivated (having at least some degree of viscosification or solidification) PEA (111) has fully filled the aneurysmal space (200) and solidified within. Depicted in FIGS. 1a-1f, target vessels are considered as, but not limited to, 200, 203, 204, 205.

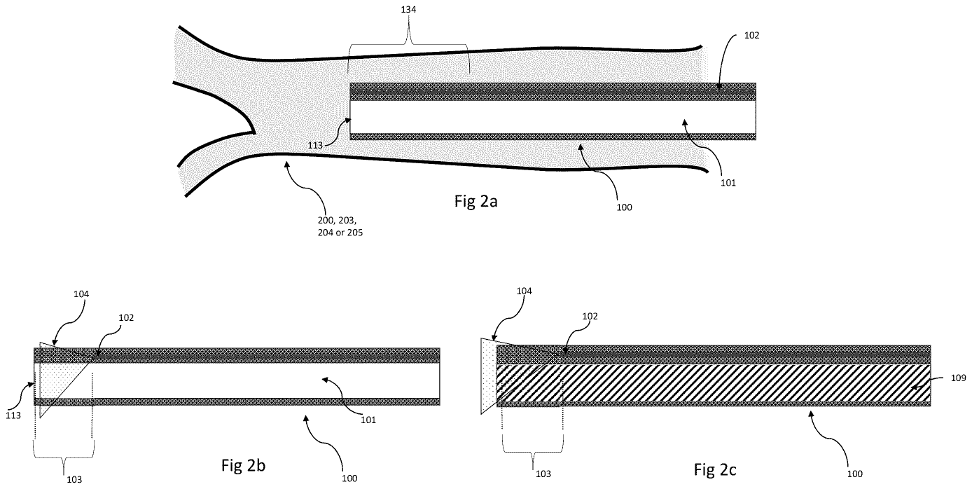

FIG. 2a depicts an example of the distal end of the microcatheter device (100) in its simplest form contained in a target vessel (200, 203, 204, or 205). It is comprised of a working lumen (101) configured to provide intravascular injection of a Photoactivated Embolic Agent (PEA) via at least one outlet port (113). It also comprises a mechanically connected waveguide (102) operably connected to the working lumen (101) and configured to emit at least one electromagnetic wave via at least one outlet port (113) for delivery to the target vessel (via delivery of the electromagnetic wave(s) to the PEA). For simplicity, not depicted in FIGS. 2a to 2c are the fluid connections of the PEA from a syringe or accessory capable of applying pressure to achieve the flow of the PEA through the working lumen (101) and out of the outlet port (113). The outlet port (113) is located at the distal end (134) of the working lumen (101). Since the PEA is delivered to a target vascular anatomy, such as target vessel (200, 203, 204, or 205) within the human anatomy, the working lumen (101) is typically large enough in diameter to fit a conventional guidewire which surgeons use to navigate to target vessel (200, 203, 204, or 205) and to also facilitate the injection of the PEA. When the target vessel is navigated to, the operator can apply hydraulic pressure to the working lumen (101) to deposit the PEA. Exposure to electromagnetic waves is necessary to crosslink and solidify the PEA in the target vessel. The electromagnetic waves are propagated along the waveguide (102) and their transmission is controlled by the operator. Delivering PEA and/or navigating with a guidewire through the working lumen provides damage to an unprotected waveguide. To limit the damage to the waveguide (102), mechanical protection is provided to the waveguide (102). All implementations of the waveguide (102) discussed here have at least some degree of mechanical protection and henceforth the waveguide will be referenced as the mechanically protected waveguide (MPW) (102).

FIGS. 2b and 2c depicts a variation of example microcatheter (100), according to non-limiting implementations. The MPW (102) is recessed from the distal end (134) to provide a pre-cure region (103) within the microcatheter device (100) and proximate outlet port (113), between the MPW (102) and the outlet port (113). FIGS. 2b and 2c shows an electromagnetic wave (104) radiating both within the pre-cure region (103), towards the outlet port (113) and outside of the outlet port (113), and into the target vessel. The pre-cure region (103) is configured to help regulate the viscosity of the PEA prior to injection into the target vessel (200, 203, 204, or 205). Recessing the MPW (102) allows for viscosification of the PEA (109) in the pre-cure region (103), and prior to entering the target vessel. Dynamically adjusting the total power of the electromagnetic waves (104) gives a level of control to the operator by controlling the viscosity of the PEA (109) as it enters the target vessel (200, 203, 204, or 205). It is understood that various disease sites will require a different viscosity. FIG. 2b depicts a scenario where there is no PEA present in the working lumen (101). Where as FIG. 2c depicts uncured PEA (109) within the working lumen.

The MPW (102) may be (but not limited to) made from a multimode fiber optic. Other suitable MPW (102) materials can include single mode optical fiber, glass rods, contained fluid channels, channels contained within reflective material, or any suitable combination thereof.

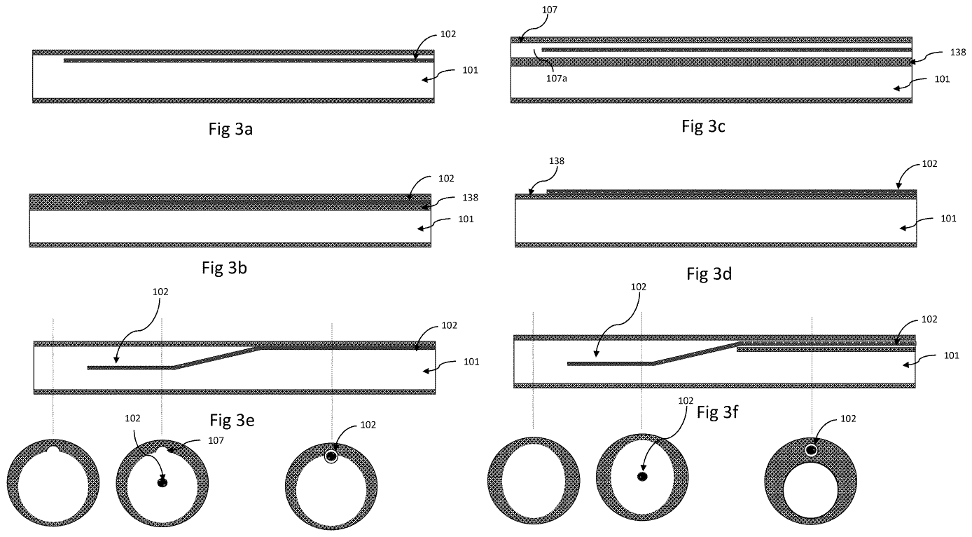

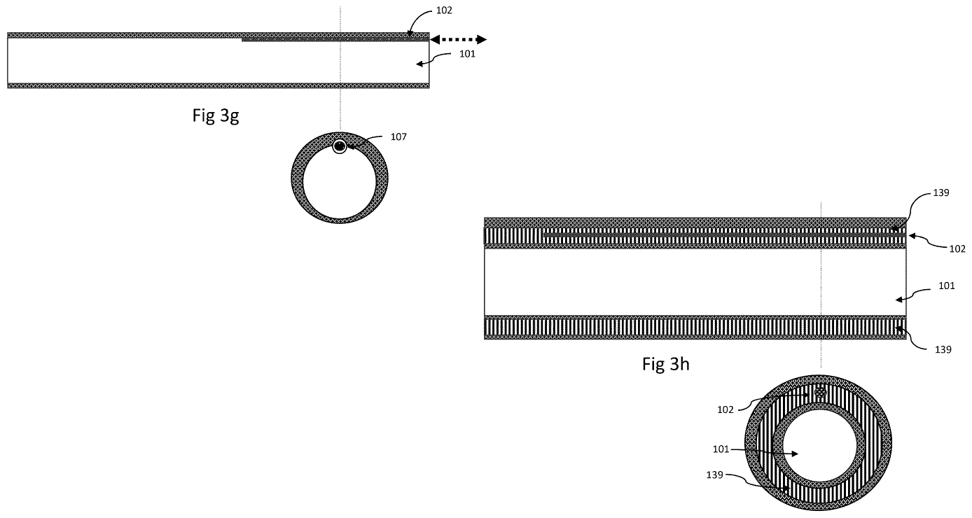

FIGS. 3a to 3 h depict a few example ways and degrees of providing mechanical protection to the MPW (102). FIG. 3a shows an example where no additional mechanical protection is provided to the MPW (102). The entire length of the MPW (102) would be in physical contact with either the PEA or a guidewire. As a result, there would be no protection from the guidewire through the normal use of the working lumen (101). FIG. 3b depicts an example way to provide mechanical protection where the MPW (102) is extruded with the wall (138) of the working lumen (101) (co-extruded). For this method, the MPW (102) is configured to withstand the temperatures necessary for lumen extrusion. Other methods include, but are not limited to, reflowing the working lumen (101) such that the MPW (102) is placed or otherwise at least partially embedded within the wall (138). FIG. 3c depicts a secondary lumen (107) within the working lumen (101) wall (138). The secondary lumen (107) is large enough to fit the MPW (102) through. In this respect, the provision of the secondary lumen (107) provides an enclosure, such as enclosure (107a), in which the MPW (102) is at least partially disposed separate from the working lumen. The dual-lumen extrusion could be manufactured, and the MPW (102) could be fed through the secondary lumen (107). The wall clearance between the MPW (102) and the inner diameter of secondary lumen (107) is usually minimal due to the size constraints of the overall microcatheter diameter. Coatings on either the inner diameter of the secondary lumen (107), or other enclosure separate from the working lumen, and MPW (102) can be used to help channel the MPW (102) through during the entire length of the working lumen (101) manufacturing process. A reflowing process, the addition of glue or heat shrinking are examples of methods for fixing the MPW (102) within the working lumen (101) such that there is no independent translation during manufacture or use. FIG. 3d depicts the MPW (102) adhered to the outer wall of the working lumen (101). Being mounted on an exterior section of the working lumen (101), such as the outer wall (138), also helps to create mechanical separation (and therefore at least some mechanical protection) between the PEA and/or guidewire. Various methods can be embolic employed to adhere it to the wall including (but not limited to) heat shrinking an additional wall, using a glue or re-flowing. With the example implementations shown in FIGS. 3b to 3d, there is generally no direct contact by the PEA and/or a guidewire with the MPW (102), which further reduces the likelihood of failure. FIG. 3h represents another example implementation of mechanical protection where the MPW (102) is woven within braided portion (139) and/or coiling layers of the microcatheter. The existence of braiding (139) and/or coiling within microcatheter is common and may pose as being obstructive to the MPW (102). However, according to some implementation, the MPW may also be woven between various braiding (139) or coiling layers. Another embodiment of mechanical protection is embedding the MPW (102) into an independent working lumen (101), with the intent of using it in coordination with the microcatheter discussed herein. In FIGS. 3e and 3f, the MPW (102) is adhered to and protected by the secondary lumen (107). Specifically for the pre-cure region, at least a portion of the MPW (102) depicted in FIGS. 3e and 3f has the ability to float within the working lumen (101) to allow for a deposit of electromagnetic waves (104) in the middle of the working lumen (101). If a solid element (such as the guidewire) is inserted to the working lumen (101), the MPW (102) will have the freedom to re-center into the secondary lumen (107) within the pre-cure region (103).

FIG. 3g shows another example mechanism for mechanical protection to the MPW (102), where it has the ability to be retracted or moved in to the secondary lumen (107) while a solid element is inserted into the working lumen (101). The MPW (102) could be moved back into the working lumen once the solid element is reinserted.

The microcatheter's working lumen (101) may be comprised of biocompatible materials. Examples of suitable biocompatible materials are Nylon, Vestamid™, Pebax™ and Polytetrafluoroethylene (PTFE). Any suitable biocompatible materials are also contemplated.

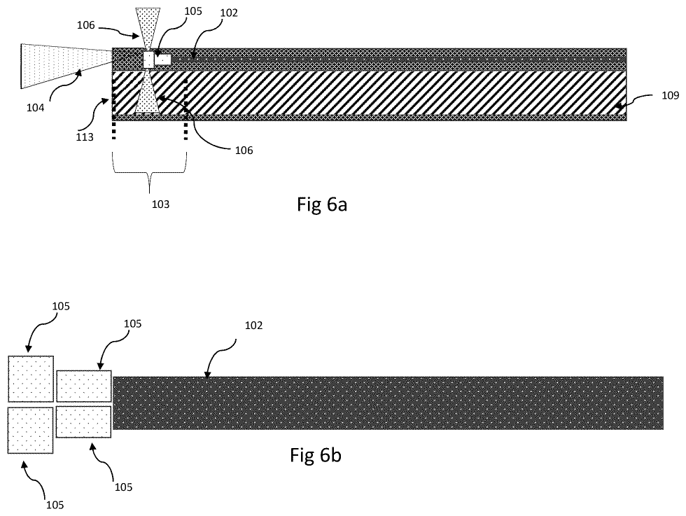

The MPW (102) can be configured to emit electromagnetic waves (104) in various directions (see, for example, FIG. 6). According to some implementations, electromagnetic waves (104) can be emitted towards the outlet port (113), through the outlet port (113) towards the target vessel, such as target vessel (200, 203, 204, or 205), towards the pre-cure region (103), and/or radially towards the target vessel. These locations may be referred to herein as (but not limited to) target regions. According to some implementations, MPW (102) can be physically oriented and fixed in place with, for example, either glue epoxy or reflowing techniques.

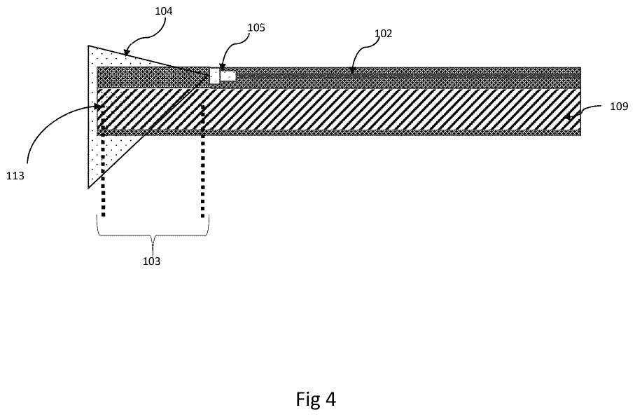

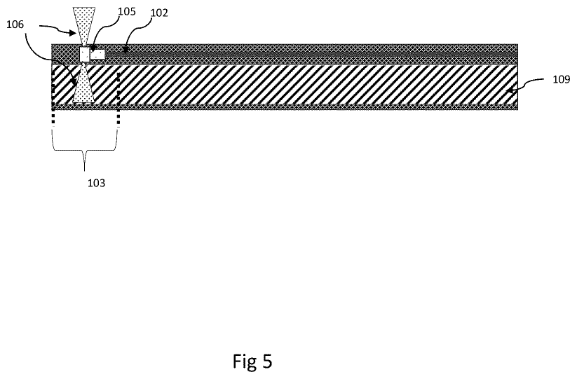

Additionally, at least one beam-forming element (for example, micro-optical components) (105) could be added at any suitable location along the optical path length of the MPW (102) to further assist with directing the electromagnetic wave (104) toward any of the target regions. The MPW (102) will emit light based on the Numerical Aperture (NA). Adding beam-forming elements (105) may help provide capability for further directing light toward the target region(s). The use of multiple beam-forming elements (105) may be necessary for more complex beam profiles and directions. FIG. 4 shows an example of the beam-forming elements (105) being used to expand the electromagnetic wave to get coverage within the pre-cure region (103) as well as towards and past the outlet port (113). In this figure, the orientation of the MPW (102) includes the location at which the MPW (102) is recessed, which can provide better coverage into the pre-cure region (103). Alternatively conceptualized, the MPW (102) can be oriented further into the working lumen (101) which also increases the pre-cure region (103) size. Said beam-forming elements (105) can either be mechanically attached to the MPW (102) or can be physically separated from the MPW (102) as long as the electromagnetic waves (104) are affected by the beam-forming elements (105).

The example microcatheter depicted in FIG. 5 also utilizes beam-forming elements; however, the electromagnetic waves are directed towards the pre-cure region and/or radially outwards. Examples of suitable beam-forming elements (105) that directs electromagnetic waves in one or more directions include, but are not limited to, a prism or folding mirror operatively coupled to a focus or defocusing component. According to some implementations, the at least one beam-forming elements comprise one or more optical components, such as one or more of a lens, a diffuser, a filter, a reflector, a prism, a folding mirror and a mask. Any suitable beam-forming elements or combination thereof is contemplated.

FIG. 6a also depicts beam-forming elements (105), however the electromagnetic waves (104 and 106) are directed towards the pre-cure region (103) for delivery thereto, towards the outlet port (113), towards the target vessel, and/or radially (at least one radial emissive beam is provided for delivery to the pre-cure region). In addition to a prism, folding mirrors, focusing/defocusing elements, and/or beam splitters can be used, where a pre-determined splitting ratio to best optimize cross-linking in the PEA (109).

According to some implementations, the at least one beam forming element is configured to provide at least a first emissive beam directed towards the target vessel and a second emissive beam towards the pre-cure region. (See, for example, FIG. 6a.)

According to some implementations, the at least one beam-forming element comprises a plurality of beam-forming elements configured to provide a plurality of radial emissive beams in a plurality of directions. (See, for example, FIG. 6b.)

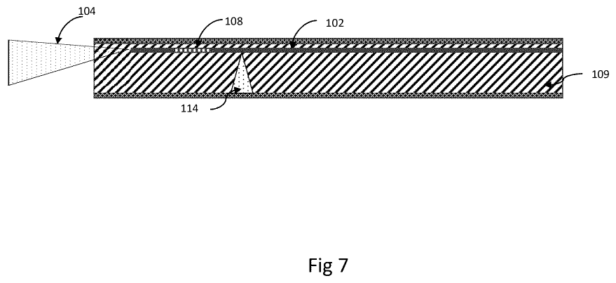

According to some implementations, the MPW (102) comprises a Fiber Bragg Grating (FBG). FIG. 7 depicts a FBG (108) disposed along the length of the MPW (102). The FBG (108) is a device that is configured such that it will reflect a specific bandwidth of electromagnetic waves while allowing the remaining spectrum of electromagnetic waves to be transmitted. According to some implementations, the FBG is tuned to a secondary wavelength and is configured to back-reflect light having the second wavelength. For example, the FBG (108) could be configured such that it reflects a spectrum of 22. When electromagnetic waves of 22 are propagated through the MPW (102), the large majority of it is usually reflected and exits the MPW (102) as a secondary cure wavelength (114). According to some implementations, the FBG is configured to selectively propagate light of a first wavelength in a first direction and to propagate light of a second wavelength in a second direction based on a frequency of the electromagnetic wave. For example, if a spectrum of electromagnetic waves, λ₁, (such as electromagnetic waves in the Ultraviolet range) is propagated and whose spectral profile does not overlap with λ₂, it will usually transmit past the FBG (108) and in a different direction than the secondary cure wavelength (114). When the microcatheter is configured with a back-end laser that is capable of providing both 21 and 22, the FBG (108) can provide a means to unequally distribute the amount of total emitted electromagnetic waves (104, 114) in various directions (104 being a non-limiting example of light directed in a first direction towards the outlet port and 114 being a non-limiting example of light directed in a second direction in a radial direction and vice-versa). Furthermore, if the back-end laser has the ability to select λ₁ and/or λ₂, the operator has the ability to select which emitting electromagnetic wave (104 or 114) to propagate to the PEA (109). The filter mechanism presented here is not limited to a FBG by a spectral filter component. Any suitable spectral component in place of or in combination with the FBG is contemplated.

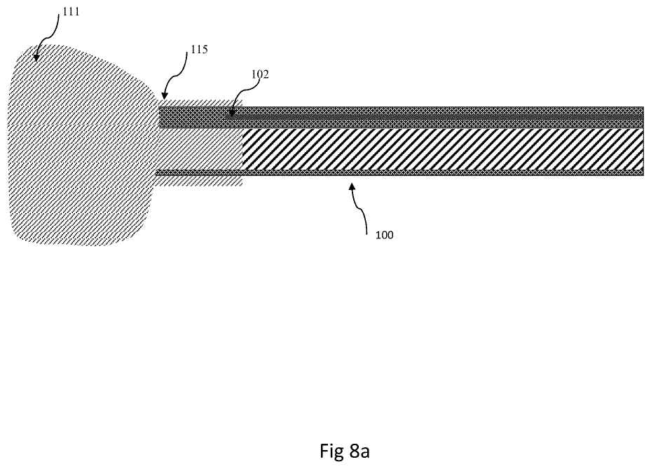

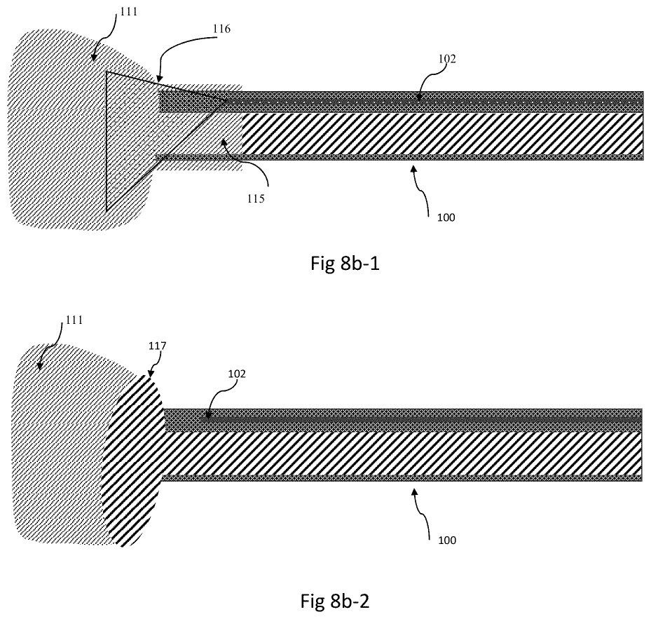

According to some implementations, microcatheter (100) further comprises a detachment mechanism configured to detach solidified PEA from a distal end of the microcatheter. FIGS. 8a to 8b-2 depict a detachment mechanism where a photo-dissolving wavelength (116) is used to dissolve cured PEA (111) to release a stuck microcatheter (100) from a cured PEA (115). FIG. 8a shows the microcatheter embedded and stuck within a fully cured PEA (111). A portion of the fully cured PEA (115) is unintentionally adhering to the microcatheter (100) and removal of the microcatheter (100) would detach the PEA (111) from a target vessel. According to some implementations, the detachment of the PEA is induced by emitting through the MPW a predetermined wavelength of electromagnetic waves configured to photodegrade the crosslinked PEA (e.g., via a photodegradation mechanism operatively connected thereto). FIG. 8b-1 shows a photo-dissolving wavelength (116) that is a different spectrum than the electromagnetic wave used to cure the PEA (104, 106, and/or 114). FIG. 8b-2 shows the result of the photo-dissolving wavelength (116) which was able to break down the cured PEA (115) back into an uncured PEA (117). This will release the embedded microcatheter (100) from the cured PEA (115). The photo-dissolving wavelength (116) can also be directed in multiple directions as necessary.

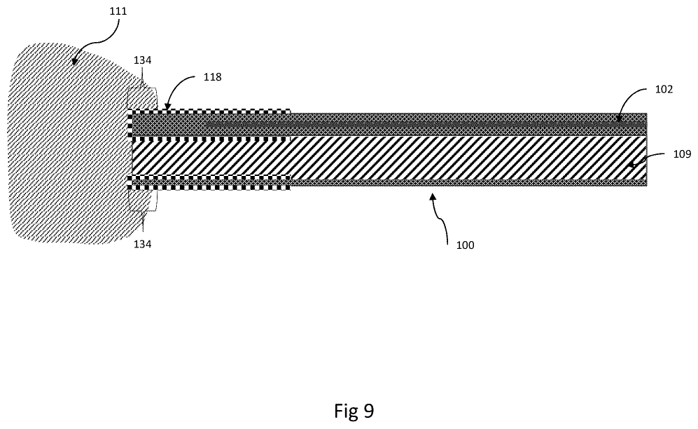

According to some implementations, the detachment mechanism comprises an ultra-hydrophobic coating applied to an exterior surface of the distal end proximate the at least one outlet port. FIG. 9 depicts an example mechanism for reducing the occurrence of a microcatheter (100) being embedded into a cured PEA (111), according to non-limiting implementations. A portion of the distal end (134) of the microcatheter that is expected to be unintentionally embedded into the cured PEA (111) is coated with a hydrophobic coating (118). The portion of the distal end (134) can also include the pre-cure region (103). It will be generally understood by one skilled in the art that such a hydrophobic coating may comprise of non-limiting examples such as silica-based materials and fluoropolymers. A hydrophobic coating usually reduces the likelihood of contact between the PEA (111) and the microcatheter (100) material. It is typical of the large majority of microcatheters on the market to have a hydrophilic coating for the entire length to ease insertion into the body when wet. However, according to some implementations, usually only the very distal end of microcatheter (100) (such as the distal end of the working lumen and the outer surface of the distal end of the microcatheter) can be hydrophobic as to not interfere with the necessary hydrophilic coating with the large majority of the microcatheter. Additionally, the coating (118) could be of a material type that does not adhere to the key bonding element in the uncured and cured PEA (109, 111). The coating can either be applied to the material or the entire region can be comprised of said coating.

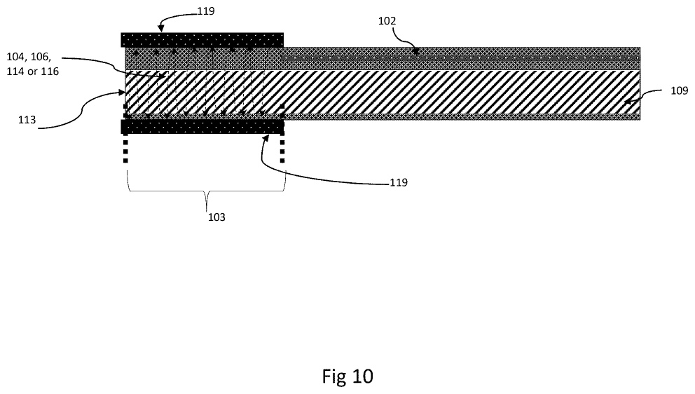

According to some implementations, at least a portion of the distal end of the working lumen comprises a reflective coating and/or reflective material configured to reflect excess light emitted radially outward in a radially inward direction. FIG. 10 shows a non-limiting implementation where an optical reflector (119) is applied around some portion of the pre-cure region (103). The optical reflector (119) behaves as an optical cavity where electromagnetic waves (104, 106, 114 or 116) exiting the pre-cure region (103) are radially reflected inwards towards the pre-cure region (103) again. This helps ensure a higher efficiency within the pre-cure region (103) and directs a larger number of electromagnetic waves (104, 106, 114, 116) toward the outlet port (113). An example of an optical reflector (119) is a metallic material like a radio-opaque marker band. Other suitable modes of providing an optical cavity operatively connected to the pre-cure region include, but are not limited to, coating the inner surface of the lumen with a reflective metallic coating such as gold, chromium, and platinum through processes like vapour deposition.

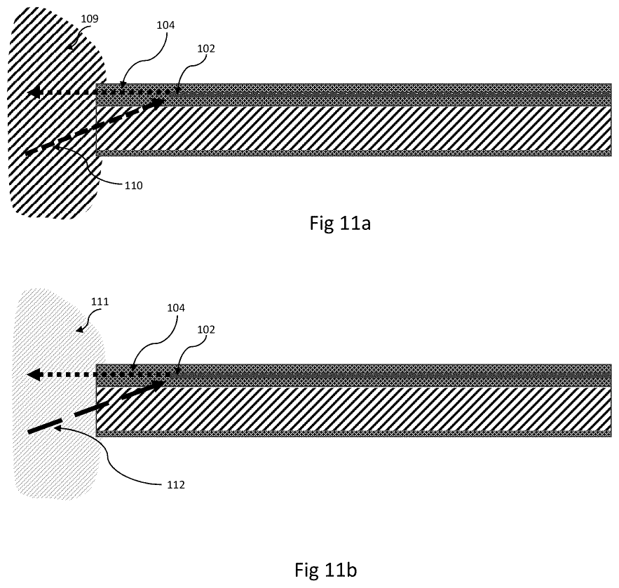

Attention is now directed to FIGS. 11a and 11b. In FIG. 11a, the microcatheter has injected the PEA (109) into the target vessel. The electromagnetic wave (104) is transmitted to the PEA (109) to induce a cure or viscosity change. There will be reflected electromagnetic waves from uncured PEA (110) channeled back into the MPW (102). Within the back-end (not depicted) an electromagnetic wave sensor can create a baseline reading based on either of power, spectrum or polarization. FIG. 11b, shows a state of the fully cured PEA (111) and the reflected electromagnetic waves from cured PEA (112) channeling into the MPW (102). Within the back-end (not depicted) an electromagnetic wave sensor will compare the measurement to the baseline reading. This will be an indicator of relative cure-ness to the operator. Observation of the PEA (109 or 111) state can also apply when detecting the dissolution of the PEA (111) during a stuck microcatheter removal. Properties observed included back-reflected power, polarization state and wavelength.

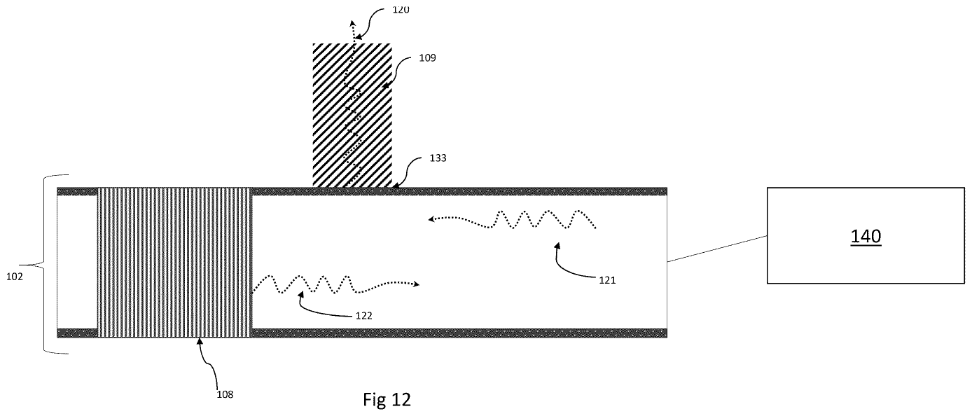

According to some implementations, the MPW comprises a fiber optic element configured to direct at least one evanescent wave to be in physical contact with the PEA. According to some implementations, the optical index of refraction can be detected in the PEA. FIG. 12 demonstrates an example embodiment of the MPW (102) wherein a portion of it is physically in contact (133) with the PEA (109). The MPW (102) can transmit a wavelength specific for evanescent function, known as evanescent wave source (121). If a small portion of the MPW (102) is exposed to the PEA (109), there will be leakage of the evanescent wave source (121) into the PEA (109) due to the evanescent wave phenomenon. The leaked portion is the evanescent wave (120). The intensity of the evanescent wave is directly related to the optical index of refraction of the PEA (109) that is in physical contact (133). Also embedded in this embodiment is a FBG (108) or other fiber optic element that is configured specifically to reflect the electromagnetic wave of the evanescent wave source (120). The reflected evanescent wave source (122) intensity is directly correlated to the intensity of the evanescent wave (120). Therefore, if the intensity of the evanescent wave (120) were to increase, it would cause the reflected evanescent wave source (122) to decrease, and vice versa. Given that the evanescent wave (120) is related to the index of refraction of the PEA (109), the measurement of the relative intensity of the reflected evanescent wave source (122) and the known evanescent wave is an indicator of the index of refraction of the PEA (109). A possible feature of the PEA (109) is its index of refraction change based on its viscosity and/or its cure state.

According to some implementations, microcatheter (100) further comprises a detecting mechanism (140) for measuring the changed optical power, polarization state and/or wavelength configured to detect changes in the optical index of refraction of a medium surrounding the mechanically protected waveguide.

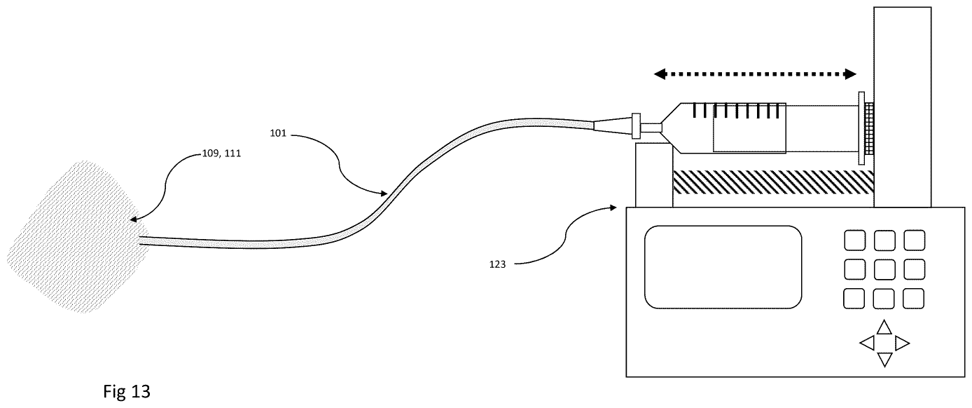

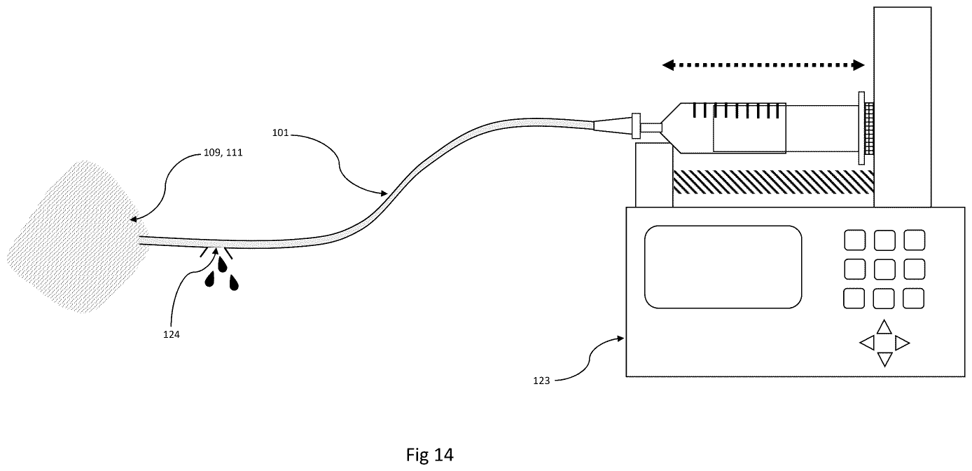

According to some implementations, microcatheter (100) further comprises a power injector configured to provide a fluid to the outlet port at a pre-determined pressure and to determine changes in a backpressure of the fluid. FIG. 13 demonstrates an example configuration of the microcatheter where it is hydraulically attached to a power injector (123) capable of detecting backpressure of the PEA (109,111). A power injector (123) can inject the PEA (109) at a given rate and internal pressure of the system. As the PEA transitions from an uncured state (109) to a cured state (111), the power injector (123) will provide the same force onto the hydraulic system. If the internal pressure were to increase past a particular relative threshold, it would usually indicate there is some blockage within the microcatheter or solidification at the distal end of the working lumen (101). This is an indirect method for detecting the viscosity of the PEA (109, 111). This can then be reported to the operator. An alternative example configuration is shown in FIG. 14, where a break or leak (124) is introduced anywhere within the hydraulic system (including the working lumen (101)). The break would cause a sudden drop in the internal pressure and the power injector (123) would usually be able to detect this and report to the operator. According to some implementations, the determination of changes in the back pressure of the fluid is made in real-time or near real-time.

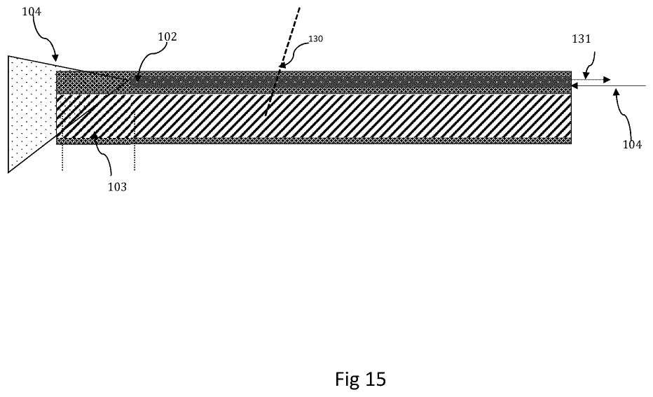

Attention is now directed to FIG. 15 for an example of the detection of a failure scenario within the MPW (102). The electromagnetic waves (104) are propagated within the MPW (102) and small amount of back-reflected electromagnetic waves (131) can be detected along the same line. If any imperfections or breakage (130) is introduced onto the MPW (102), a reflection point is created. The intensity of the back reflected electromagnetic waves (131) will increase in intensity. Monitoring a relative increase in intensity of back reflected electromagnetic waves (131) can indicate a breakage (130) on the MPW (102)

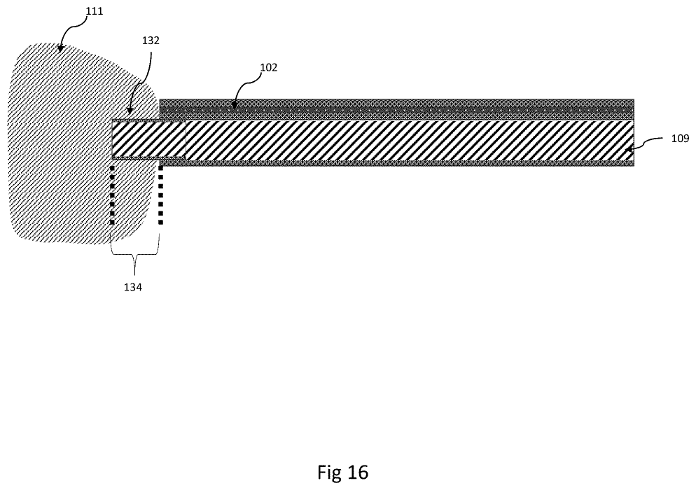

Another example implementation of the described microcatheter device is where the distal end of the microcatheter is detachable from the working lumen is shown in FIG. 16. In a scenario where the PEA has been fully cured (111), the distal end (134) of the microcatheter may be encapsulated and not easily removable. The distal portion can have a detachable tip (132). Some methods to enable detachment of the tip from the microcatheter include some of the following, individually or in any suitable combination: physically separating by force such that a pre-determined tear point is broken, physically separating by dissolving the pre-determined tear point by the emitted electromagnetic wave, physically separating by dissolving the pre-determined tear point through the use of a biocompatible injectable gel specifically designed for dissolving. Furthermore, the detachable tip (132) would remain in the cured PEA (111) and subsequently in the target vessel. The detachable tip (132) would be made of or otherwise comprise a biocompatible material that would naturally dissolve over time when in the presence of the bloodstream. According to some embodiments, the detachable tip is configured to dissolve within the target vessel over a predetermined amount of time.

According to some implementations, a distal end of the MPW is free to float within the pre-cure region, as discussed further below.

According to some implementations, the microcatheter (100) further comprises at least one valve mechanism, as discussed further below.

According to some implementations, the distal end of the working lumen is tapered. This would increase the local resistance to the flow of PEA, and thus under a constant-pressure driving regime (such as when the operator is applying a constant force to the syringe plunger), reduce the flow rate of the PEA. Coupled with an implementation of a pre-cure region, this local flow rate reduction can in term increase the time of exposure to EM energy emitted by the MPW and thereby improve the viscosification/solidification efficiency and prevent accidental release of uncured PEA into the anatomy.

According to some implementations, the microcatheter (100) further comprises a solid element inserted into the working lumen, such as the guidewire.

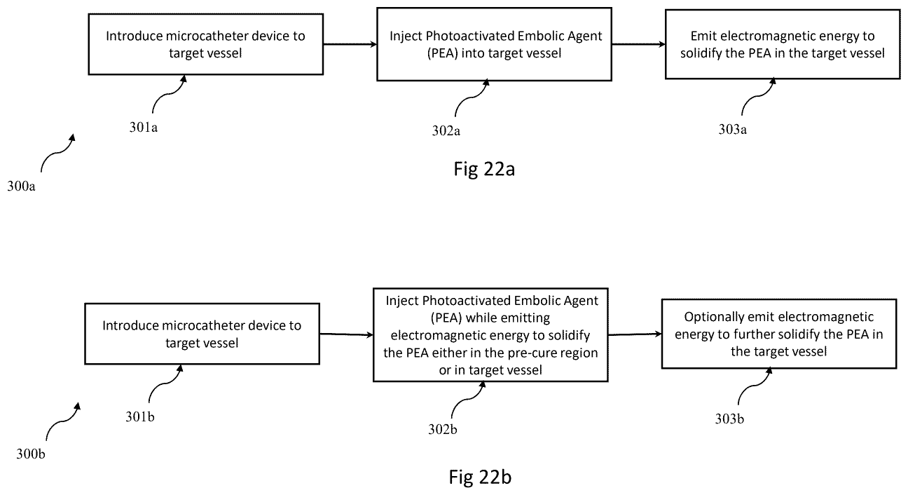

FIGS. 22a and 22b depict two non-limiting methods 300a and 300b of endovascular embolization using the microcatheter devices described herein. In the first case, the operator typically begins by performing necessary endovascular navigation to reach the target vessel. Once the catheter is correctly placed within the target vessel, the operator shall inject the PEA without having yet switched on the EM energy source. Subsequently, once the target vessel is sufficiently filled with the uncured PEA, the operator shall enable the EM energy source to be emitted through the distal region of the microcatheter device, causing the PEA to solidify. In the second case, similarly, the operator shall perform necessary endovascular navigation to reach the target vessel. Once the optimal positioning of the microcatheter device is confirmed, the operator shall enable the electromagnetic energy source simultaneously with the time of PEA injection, such that the PEA is being viscosified or solidified as it exits through the distal region of the microcatheter device. The first method 300a may be referred to as the sequential approach while the second method 300b may be referred to as the concerted or simultaneous approach. The sequential method might typically be preferred in cases wherein the local blood flow rate is relatively low and/or in cases where maximum penetration and shape-conformability of the PEA to the treatment site is desired (such as in the case of a hypervascular tumor comprising of many capillary vessels). The simultaneous method wherein the PEA is either viscosified or solidified at the time of injection will decrease the shape-conformability of the PEA to various blood vessels, but may be preferred in cases where there are critical structures downstream and the PEA must not over-penetrate the treatment site, which would risk ischemia in the critical structure (such as an arteriovenous malformation).

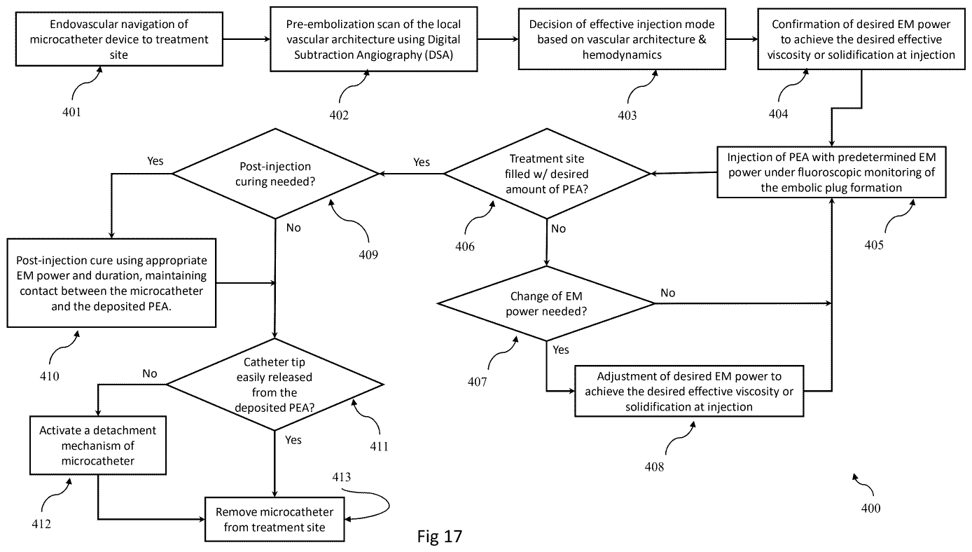

Attention is now directed to FIGS. 17, 22a and 22b which depict flowcharts of methods (300a), (300b) and (400) of endovascular embolization using the microcatheter devices described herein, according to non-limiting implementations. The following discussion of methods (300a), (300b) and (400), will lead to a further understanding of the devices described herein. However, it is to be understood that methods (300a), (300b) and/or (400) can be varied and need not work exactly as discussed herein in conjunction with each other, and that such variations are within the scope of present implementations. For example, methods (300a), (300b) and/or (400) need not be performed in the exact sequence as shown, unless otherwise indicated; and likewise various blocks may be performed in parallel rather than in sequence; hence the elements of methods (300a), (300b) and (400) are referred to herein as “blocks” rather than “steps”.

Attention is directed to FIG. 22a. At block 301a, the microcatheter device, such as microcatheter device 100, is introduced to the target vascular anatomy, such as target vessel (200, 203, 204, or 205). At block 302a, the PEA (109) is injected into the target vascular anatomy until a desired volume of the target vascular anatomy has been filled. At block 303a, after the injection, at least one electromagnetic wave is emitted to the PEA disposed in the target vascular anatomy at one or more solidification wavelengths of the PEA.

According to some implementations, prior to emitting the at least one electromagnetic wave at the at least one solidification wavelength, method 300a further comprises determining whether there has been a failure of the microcatheter device (for example, as discussed above). According to some implementations, this determining block comprises sensing at least one electromagnetic wave that is back-reflected from the MPW.

According to some implementations, method 301a further comprises, after emitting the at least one electromagnetic wave at the solidification wavelength (block 303a), removing the microcatheter from the solidified PEA and the target vascular anatomy. According to some implementations, removing the microcatheter comprises detaching the microcatheter from the solidified PEA. According to some implementations, detaching the microcatheter comprises activating a photodegradation mechanism.

According to some implementations, method 300a further comprises determining a solidification state of the PEA. According to some implementations, the determination is performed in real-time or near real-time (typically latency of less than one second) with the emission of the at least one electromagnetic wave to the PEA (block 303a). According to some implementations, the determination comprises detecting at least one electromagnetic wave that is back-reflected from the PEA disposed in the target vascular anatomy.

Method 300b (FIG. 22b) shares the action of block 301a (introducing the microcatheter to the target vascular anatomy). However, at block 302b, the PEA is injected into the target vascular anatomy while (simultaneously) the at least one electromagnetic wave at one or more solidification wavelengths is emitted to solidify the PEA in either the pre-cure region or the target vascular anatomy. At block 303b, optionally, at least one electromagnetic wave is emitted to further solidify the PEA disposed in the target vascular anatomy. Similarly to method 300a, method 300b further comprises determining a solidification state of the PEA. For example, the determination of the PEA's solidification state may be performed in real-time or near real-time with the emission of the at least one electromagnetic wave to the PEA (blocks 302b and 303b). According to some implementations, the determination comprises detecting at least one electromagnetic wave that is back-reflected from the PEA disposed in the target vascular anatomy.

Attention is once again directed to FIG. 17 and the individual blocks comprising the method (400). A typical use of the microcatheter typically includes navigating the microcatheter to the treatment site using a conventional microguidewire (block 401), which may further be guided by a larger gauge support catheter. Once at the treatment site, pre-embolization scans are typically performed using digital subtraction angiography (block 402). The digital subtraction angiography provides the local vessel size/diameter, blood flow rates, downstream angio-architecture and the presence of any critical structures. According to the prior knowledge of the anatomy as well as the observations made during the digital subtraction angiography, the operator first decides whether the PEA injection will be carried out via the concerted or the sequential method, which changes the timing of the electromagnetic energy emission with respect to the timing of the injection. Subsequently, the operator could decide what degree of viscosification or solidification is necessary for the PEA to optimally fill the treatment site (the target vascular anatomy) based on such factors affecting the local fluid dynamics (block 403). The operator(s) would then decide and select the correct electromagnetic power output for the desired viscosity (block 404). In the case of sequential method, the chosen EM power output is nominally zero. The operator will inject the PEA to the treatment site until it is filled (block 405). Using live fluoroscopy or additional digital subtraction angiography, the operator can confirm when the treatment site is filled and repeat or continue the injection as needed (block 406). It is noted that at any point during the injection, the operator may choose to change the electromagnetic power output based on the fluoroscopic feedback (blocks 407 and 408). Thus, it is possible that the operator initially begins the injection using the sequential approach (i.e. no simultaneously emitted electromagnetic energy during PEA injection) but decides to enable the emission of electromagnetic energy halfway through the injection to viscosify or solidify the subsequently injected PEA. Such a situation might occur if the operator observes excessive PEA wash-away due to high blood flow, for example. Once the treatment site is filled with the desired amount of PEA, the operator decides whether to perform a post-injection curing (block 409). If post-injection curing is deemed necessary, the operator can decide and select the correct electromagnetic output power as well as the duration. The post-injection curing is then performed by enabling the electromagnetic energy source while maintaining the microcatheter position within the vessel such that the distal end of the microcatheter device is in physical contact with the deposited PEA, as confirmed through fluoroscopy (block 410). It is noted that there are several methods describe here to monitor the solidification of the PEA such as the detection of evanescent waves through an integrated FBG using some embodiments which are not shown in this flowchart. After solidifying the PEA, the operator can slowly retract the microcatheter device from the treatment site and sense if there is any resistance to removal, for instance due to the adhesion between the solidified PEA and the microcatheter tip (block 411). If there are difficulties in removing the microcatheter device (such as when observing that pulling on the microcatheter device results in PEA dislodgement), the operator can activate a detachment mechanism discussed herein (block 412). Once it is deemed safe by the operator, the microcatheter device is retracted and completely removed from the body, concluding the treatment (block 413). It is noted that digital subtraction angiography can be performed at any point throughout the course of treatment (not mentioned in the flowchart) when deemed necessary by the operator to further assess the vascular structure of the treatment site and/or to confirm the integrity of the deposited PEA. Throughout the entire use of the microcatheter, there may be some fault detection mechanisms which are monitored but not mentioned in the flowchart. This includes, but is not limited to, the monitoring of the catheter burst via injection force sensing by the syringe pump, the detection of MPW breakage through detection of back-reflected light and its power, polarization states, and wavelengths.

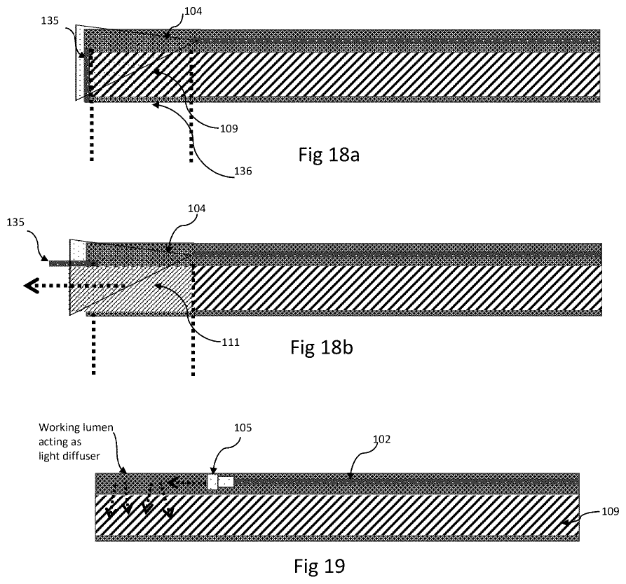

FIGS. 18a and 18b depict a configuration wherein an outlet relief valve (135) is integrated to controllably pressurize the pre-cure region and restrict the flow of PEA (109), according to non-limiting implementations. Such a mechanism might be employed in order to ensure consistent viscosification/solidification of the injected PEA (109) and limit accidental release of uncured PEA (109), which can result in non-target embolization. FIG. 18a depicts a configuration of the pre-cure region of the microcatheter device with outlet valve (135) in a closed state. Outlet relief valve is configured to open when a predetermined relief pressure (typically in the order or one tenth to hundreds of PSI) within the pre-cure region is reached. With the outlet relief valve (135) closed, the PEA (109) flow within the pre-cure region is stagnated, allowing more time for a given volume of PEA (109) to be exposed to the electromagnetic energy (102). Once sufficient viscosification or solidification of PEA (109) within the pre-cure region (103) is achieved, the cured PEA (111) can be released into the target vessel simply by applying a sufficient pressure to open the outlet relief valve (135). The outlet relief valve may close again and the process may repeat multiple times over the course of the treatment.

FIG. 19 depicts an example configuration for diffusing the electromagnetic wave (104) through the working lumen (101). Diffusing the electromagnetic waves (104) is beneficial as it would allow for more efficient absorption from the uncured PEA (109) over a controlled distance, irrespective of the electromagnetic energy absorption by the PEA compared to configurations which have forward-firing regimes. The working lumen (101) could be doped with a scatterer, or the material selection would be made such that it scatters the emitted electromagnetic wave(s) (104), acting as a light diffuser.

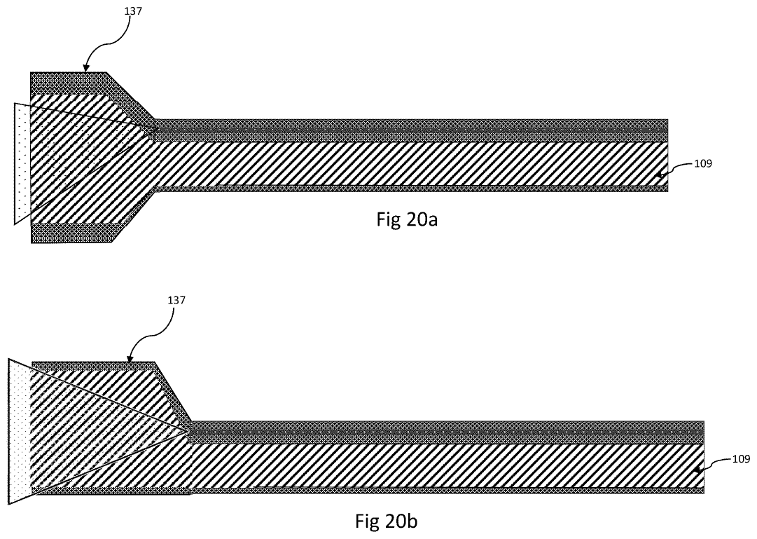

FIGS. 20a and 20b depict non-limiting configurations of the working lumen (104) where the pre-cure region is widened (103). Having a widened precure region (137) effectively increases the cross-sectional area and decreases the flow velocity of the uncured PEA (109) under a constant flow rate regime, such as when the injection is being driven by an infusion pump. With a decrease in flow velocity, the time in which the electromagnetic waves (104) have to be absorbed by the uncured PEA (109) is increased therefore allowing for a higher solidification rate. In the scenario where a completely cured PEA (111) is ejected through the outlet port (113), the widened pre-cure region (137) is designed such that the cured PEA (111) will have a desired shape (for example, a wider diameter). FIG. 20a shows a configuration where the widened pre-cure region (137) sits concentrically with the working lumen (101) with the MPW (102) off-center, whereas FIG. 20b depicts a widened pre-cure region (137) which has an inner diameter that sits concentrically with the MPW (102).

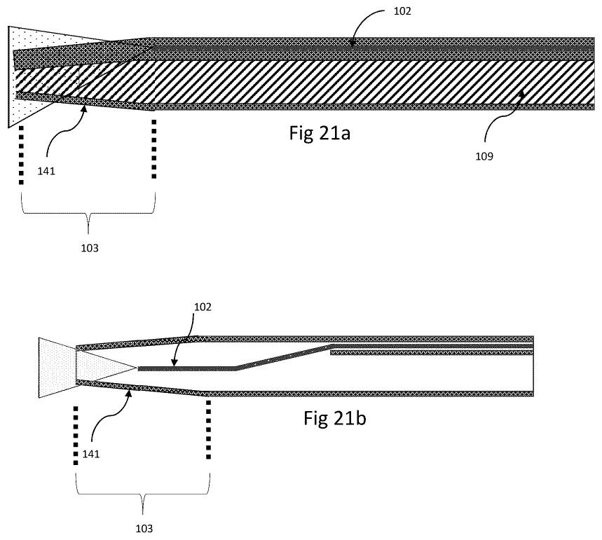

FIGS. 21a and 21b, conversely to FIGS. 20a and 20b, depict non-limiting configurations of the pre-cure regions (103) with a tapered wall (141), resulting in a smaller cross-sectional area at the tip. The tapered wall (141) effectively increases the flow resistance within the pre-cure region (103) and pressurizes it, decreasing flow rate of the uncured PEA (109) when the injection is being driven under a constant pressure regime, such as when performing a steady hand injection. FIG. 21a shows a configuration of the tapered pre-cure region (103) with the MPW (102) off-center, whereas FIG. 21b depicts a tapered pre-cure region (103) which has an MPW (102) that is positioned concentrically within the pre-cure region (103). Both tapering designs exemplified through FIGS. 20a to 21b can help achieve a more robust and consistent viscosification/solidification and reduce the occurrences of accidental release of uncured PEA (109), which can result in non-target embolization. The choice of design may depend on non-limiting factors including whether the injection is volume-controlled or pressure-controlled.

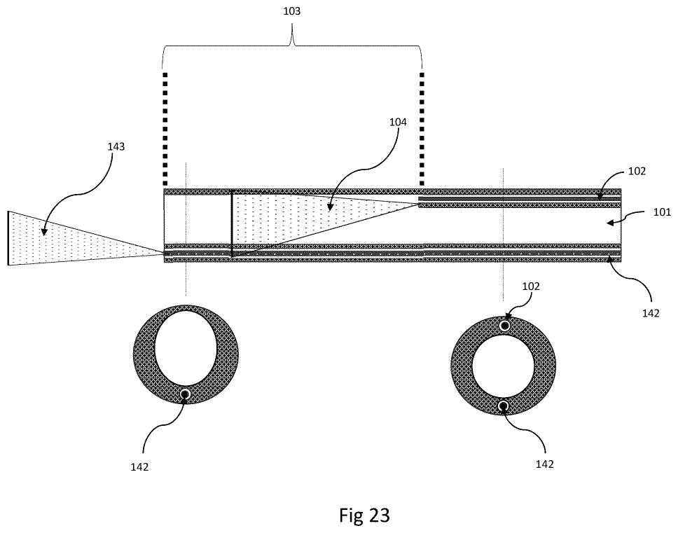

FIG. 23 depicts an example of multiple MPWs housed within respective channels and are terminated at various positions to allow for multiple beam directions, according to non-limiting implementations. In this specific example, one of the MPWs (102) is terminated immediately proximal to the pre-cure region (103) while the additional MPW(s) (142) extends to the tip of the microcatheter device. According to non-limiting implementations, either MPW (102) or MPW (142) can be individually activated or both can be activated simultaneously. To highlight one use case example, an operator may initially wish to perform an injection of PEA while simultaneously solidifying it by the emission of electromagnetic energy (104) through the MPW (102), positioned such that the pre-cure region (103) is efficiently irradiated. Once the injection is complete and the treatment site is filled, the operator may further wish to reinforce the injected PEA by further performing a post-injection cure by the by the emission of electromagnetic energy (143) through the MPW (142), positioned to effectively irradiate the intravascular space near the tip of the microcatheter device.

Persons skilled in the art will appreciate that there are yet more alternative implementations and modifications possible, and that the above examples are only illustrations of one or more implementations. The scope, therefore, is only to be limited by the claims appended hereto.

### Interpretation

It will also be understood that for the purposes of this application, “at least one of X, Y, and Z” or “one or more of X, Y, and Z” language can be construed as X only, Y only, Z only, or any combination of two or more items X, Y, and Z (e.g., XYZ, XYY, YZ, ZZ).

In the present application, components may be described as “configured to” or “enabled to” perform one or more functions. Generally, it is understood that a component that is configured to or enabled to perform a function is configured to or enabled to perform the function, or is suitable for performing the function, or is adapted to perform the function, or is operable to perform the function, or is otherwise capable of performing the function.

Additionally, components in the present application may be described as “operatively connected to”, “operatively coupled to”, and the like, to other components. It is understood that such components are connected or coupled to each other in a manner to perform a certain function. It is also understood that “connections”, “coupling” and the like, as recited in the present application include direct and indirect connections between components.

References in the application to “one embodiment”, “an embodiment”, “an implementation”, “a variant”, etc., indicate that the embodiment, implementation or variant described may include a particular aspect, feature, structure, or characteristic, but not every embodiment, implementation or variant necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely”, “only”, and the like, in connection with the recitation of claim elements or use of a “negative” limitation. The terms “preferably”, “preferred”, “prefer”, “optionally”, “may”, and similar terms are used to indicate that an item, condition or step being referred to is an optional (not required) feature of the invention.

The singular forms “a”, “an”, and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

1-44. (canceled)

45. A microcatheter device comprising:
(a) a working lumen having at least one outlet port at a distal end for intravascular injection of a photoactivated embolic agent (PEA); and
(b) a mechanically protected waveguide operably coupled to the working lumen and configured to emit electromagnetic radiation via the outlet port;
wherein the waveguide is at least partially embedded in, co-extruded with, or separated by a barrier from the working lumen, and is protected from mechanical damage during navigation and infusion.

46. The device of claim 45, further comprising a pre-cure region proximate the outlet port, configured to expose the PEA to electromagnetic radiation to increase viscosity prior to vascular injection.

47. The device of claim 45, wherein the waveguide comprises a multimode optical fiber.

48. The device of claim 45, wherein the waveguide includes a Fiber Bragg Grating (FBG) configured to reflect a secondary wavelength for selective propagation of light in multiple directions.

49. The device of claim 45, further comprising a beam-forming element configured to direct a first electromagnetic beam to the target vessel and a second beam to the pre-cure region.

50. The device of claim 49, wherein the beam-forming element comprises at least one of: a lens, a diffuser, a reflector, and a filter.

51. The device of claim 45, further comprising a detachment mechanism at the distal end configured to reduce adhesion of cured PEA, the mechanism comprising a hydrophobic coating and/or emission of photodegradation light.

52. The device of claim 45, wherein a portion of the distal end comprises a reflective material configured to redirect radially emitted light inward.

53. The device of claim 45, wherein the waveguide is configured to emit evanescent waves in contact with the PEA to assess optical index of refraction.

54. The device of claim 53, further comprising a detection mechanism configured to analyze reflected signals for monitoring the state of the PEA.

55. The device of claim 45, further comprising a power injector capable of detecting backpressure changes during injection to infer the viscosity or solidification state of the PEA.

56. The device of claim 45, wherein the distal end of the working lumen is tapered to modulate PEA flow velocity.

57. The device of claim 45, further comprising a valve mechanism configured to limit release of uncured PEA unless a pressure threshold is met.

58. The device of claim 45, further comprising an additive liner in the pre-cure region to limit adhesion of cured PEA to internal surfaces.

59. The device of claim 45, wherein the waveguide is operable with a back-end light source providing two wavelengths: a curing wavelength and a photodegradation wavelength.

60. A microcatheter comprising:
(a) a beam-forming element disposed along a waveguide within the microcatheter, configured to emit electromagnetic radiation in both radial and forward directions; and
(b) a pre-cure region configured to receive electromagnetic exposure for viscosification of a photoactivated embolic agent (PEA) prior to exit through the outlet port.

61. The microcatheter of claim 60, wherein the beam-forming element includes a prism, folding mirror, or beam splitter.

62. A method of endovascular embolization comprising:
introducing a microcatheter device according to claim 45 into a target vessel;
injecting a photoactivated embolic agent (PEA) into the target vessel; and
either simultaneously with or after the injection, emitting electromagnetic radiation at a solidification wavelength to the PEA to initiate or complete solidification in situ.

63. The method of claim 62, further comprising detecting back-reflected light to assess solidification state of the PEA.

64. The method of claim 62, further comprising activating a photodegradation mechanism to detach the microcatheter from cured PEA.

65. A method of endovascular embolization comprising:
introducing a microcatheter device according to claim 60 into a target vessel;
injecting a photoactivated embolic agent (PEA) into the target vessel; and
either simultaneously with or after the injection, emitting electromagnetic radiation at a solidification wavelength to the PEA to initiate or complete solidification in situ.

66. The method of claim 65, further comprising detecting back-reflected light to assess solidification state of the PEA.

67. The method of claim 65, further comprising activating a photodegradation mechanism to detach the microcatheter from cured PEA.
